(19) United States
(12) Patent Application Publication
(10) Pub. No.: US 2018/0185631 A1
(43) Pub. Date: Jul. 5, 2018

(54) EXTRACORPOREAL PATCH

(71) Applicant: BLUEWIND MEDICAL LTD., Herzlia (IL)

(72) Inventor: Yossi GROSS, Moshav Mazor (IL)

(73) Assignee: BLUEWIND MEDICAL LTD., Herzlia (IL)

(21) Appl. No.: 15/395,257

(22) Filed: Dec. 30, 2016

(51) Int. Cl. A61N 1/04; A61M 37/00; A61F 13/02; A61B 34/20; A61N 1/05; A61N 1/36

(52) U.S. Cl. CPC A61N 1/0492; A61M 37/00; A61F 13/0246; A61B 34/20; A61N 1/0502; A61M 2202/0468; A61M 2205/8206; A61M 2205/587; A61M 2205/3576; A61M 2205/3303; A61N 1/36017

(57) ABSTRACT

Apparatus is provided for use with skin of a subject and with a nerve under the skin, the apparatus including a patch, having an adhesive on a first side of the patch, configured to adhere the patch to the skin. A needle including a needle cathode protrudes from the first side of the patch and punctures the skin. The apparatus also includes an anode. Control circuitry is configured to stimulate the nerve by driving pulsatile current between the needle cathode and the anode. Other embodiments are also described.

### FIELD OF THE INVENTION

The present invention relates to stimulating a nerve under skin of a subject.

### SUMMARY OF THE INVENTION

An adhesive patch is provided for use with skin of a subject and with a nerve under the skin. The patch has a needle cathode, configured to puncture the skin, protruding from one side, an anode disposed on the same side, and control circuitry configured to stimulate the nerve by driving a pulsatile current between the needle cathode and the anode. The control circuitry typically drives the pulsatile current at a frequency that is sufficiently high that the thus-stimulated nerve is inhibited from propagating nerve signals to the subject's brain.

There is therefore provided, in accordance with some applications of the present invention, apparatus for use with skin of a subject and with a nerve under the skin, the apparatus including:

a patch, having an adhesive on a first side of the patch, configured to adhere the patch to the skin;

a needle including a needle cathode, protruding from the first side of the patch and configured to puncture the skin;

an anode; and

control circuitry, configured to stimulate the nerve by driving pulsatile current between the needle cathode and the anode.

For some applications, the anode is disposed at the first side of the patch.

For some applications, the anode is disposed on a proximal portion of the needle and the needle cathode is disposed on a distal portion of the needle.

For some applications, the control circuitry includes a light emitting diode (LED), configured to emit light upon the patch being adhered to the skin of the subject, that is disposed on a second side of the patch opposite to the first side of the patch.

For some applications, the patch is shaped to define at least one window, the window positioned to allow visualizing of the needle cathode during puncturing of the skin by the needle cathode.

For some applications, the patch does not include a drug.

For some applications, the patch does not include a pharmaceutical analgesic.

For some applications, the needle is a first needle, and the apparatus further includes a second needle, the second needle (a) including the anode, (b) protruding from the first side of the patch, and (c) configured to puncture the skin.

For some applications, a distance between the first and second needles is 5-12 mm.

For some applications, the nerve is a tibial nerve of the subject, and wherein the control circuitry is configured to stimulate the tibial nerve by driving pulsatile current between the needle cathode and the anode.

For some applications, a length of the needle is 2-8 mm.

For some applications, the apparatus is for use with a wireless transmitter, and the control circuitry further includes a wireless receiver configured to receive data from the wireless transmitter.

For some applications, the control circuitry is configured to alter the pulsatile current in response to data received from the wireless transmitter.

For some applications, the wireless receiver is a Bluetooth receiver.

For some applications, the control circuitry includes at least one physiological sensor.

For some applications, the sensor includes a heart rate sensor.

For some applications, the sensor includes an accelerometer.

For some applications, the patch includes a drug for delivery to the subject through the skin of the subject.

For some applications, the drug is an analgesic.

For some applications, the control circuitry includes a user control and is configured to iontophoretically administer a dose of the drug upon the user control being activated.

For some applications, (a) the patch constantly administers the drug through the skin of the subject when the patch is adhered to the skin, and (b) the control circuitry includes a user control and is configured to stimulate the nerve upon the user control being activated.

For some applications, the adhesive is disposed at an edge of the patch, and the patch has a non-adhesive zone on the first side of the patch, in a central region of the patch.

For some applications, the needle cathode protrudes from the non-adhesive zone.

For some applications, the anode is disposed in the non-adhesive zone of the patch.

For some applications, the control circuitry includes a battery configured to power the control circuitry to stimulate the nerve for 1-7 days.

For some applications, the patch includes a tab, removably coupled to the battery, wherein the battery is configured to be usable upon removal of the tab.

For some applications, the patch is shaped to define a fold-line, the needle cathode being disposed along the fold-line.

For some applications, the patch is folded along the fold-line, and the fold of the patch along the fold-line facilitates insertion of the needle cathode into the skin prior to unfolding of the patch.

For some applications, the patch is folded along the fold-line, the first side of the patch facing outwards.

For some applications, the apparatus further includes an ultrasound transducer interface, configured to removably secure the patch to an ultrasound transducer.

For some applications, the nerve is a sciatic nerve of a subject, and wherein the control circuitry is configured to stimulate the sciatic nerve by driving pulsatile current between the needle cathode and the anode.

For some applications, the nerve is an occipital nerve of a subject, and wherein the control circuitry is configured to stimulate the occipital nerve by driving pulsatile current between the needle cathode and the anode.

For some applications, the nerve is a median nerve of a subject, and wherein the control circuitry is configured to stimulate the median nerve by driving pulsatile current between the needle cathode and the anode.

For some applications, the anode is an adhesive anode, configured to adhere the patch to the skin.

There is further provided, in accordance with some applications of the present invention, a method for stimulating a nerve under skin of a subject, the method including:

adhering to the skin an adherable patch having a needle cathode protruding from a first side of the patch and an anode disposed on the first side of the patch;

puncturing the skin with the needle cathode; and

activating control circuitry on the patch to stimulate the nerve by driving pulsatile current between the needle cathode and the anode.

For some applications, the method further includes navigating the patch to a site by visualizing the needle cathode and the skin through a window in the patch.

For some applications, puncturing the skin with the needle cathode includes:

puncturing the skin with the needle cathode while the patch is folded; and

subsequently, while the needle cathode is inside the skin, unfolding the patch and adhering the patch to the skin.

For some applications, the method further includes removing a tab, the tab being removably coupled to a battery that is disposed in the patch and is configured to be usable upon removal of the tab.

For some applications, the method further includes using ultrasound energy to locate the nerve before the adhering of the patch to the skin.

For some applications, using ultrasound energy to locate the nerve includes navigating the patch to a site of the nerve while the patch is removably secured to an ultrasound transducer.

For some applications, the patch includes a drug for delivery to the subject through the skin of the subject, the method further including iontophoretically administering the drug.

For some applications, iontophoretically administering the drug includes activating a user control coupled to the control circuitry to administer a dose of the drug.

There is further provided, in accordance with some applications of the present invention, apparatus for use with skin of a subject and with a nerve under the skin, the apparatus including:

a patch, having an adhesive on a first side of the patch, configured to adhere the patch to the skin;

a needle including a needle anode, protruding from the first side of the patch and configured to puncture the skin;

a cathode; and

control circuitry, configured to block the nerve by driving current between the needle anode and the cathode.

For some applications, the control circuitry is configured to block the nerve by driving pulsatile current between the needle anode and the cathode.

For some applications, the cathode is disposed at the first side of the patch.

For some applications, the cathode is disposed on a proximal portion of the needle and the needle anode is disposed on a distal portion of the needle.

For some applications, the control circuitry includes a light emitting diode (LED), configured to emit light upon the patch being adhered to the skin of the subject, that is disposed on a second side of the patch opposite to the first side of the patch.

For some applications, the patch is shaped to define at least one window, the window positioned to allow visualizing of the needle anode during puncturing of the skin by the needle anode.

For some applications, the patch does not include a drug.

For some applications, the patch does not include a pharmaceutical analgesic.

For some applications, the needle is a first needle, and wherein the apparatus further includes a second needle, the second needle (a) including the cathode, (b) protruding from the first side of the patch, and (c) configured to puncture the skin.

For some applications, a distance between the first and second needles is 5-12 mm.

For some applications, the nerve is a tibial nerve of the subject, and the control circuitry is configured to block the tibial nerve by driving current between the needle anode and the cathode.

For some applications, a length of the needle is 2-8 mm.

For some applications, the apparatus is for use with a wireless transmitter, and the control circuitry further includes a wireless receiver configured to receive data from the wireless transmitter.

For some applications, the control circuitry is configured to alter the current in response to data received from the wireless transmitter.

For some applications, the wireless receiver is a Bluetooth receiver.

For some applications, the control circuitry includes at least one physiological sensor.

For some applications, the sensor includes a heart rate sensor.

For some applications, the sensor includes an accelerometer.

For some applications, the patch includes a drug for delivery to the subject through the skin of the subject.

For some applications, the drug is an analgesic.

For some applications, the control circuitry includes a user control and is configured to iontophoretically administer a dose of the drug upon the user control being activated.

For some applications, (a) the patch constantly administers the drug through the skin of the subject when the patch is adhered to the skin, and (b) the control circuitry comprises a user control and is configured to block the nerve upon the user control being activated.

For some applications, the adhesive is disposed at an edge of the patch, and the patch has a non-adhesive zone on the first side of the patch, in a central region of the patch.

For some applications, the needle anode protrudes from the non-adhesive zone.

For some applications, the cathode is disposed in the non-adhesive zone of the patch.

For some applications, the control circuitry includes a battery configured to power the control circuitry to block the nerve for 1-7 days.

For some applications, wherein the patch includes a tab, removably coupled to the battery, and the battery is configured to be usable upon removal of the tab.

For some applications, the patch is shaped to define a fold-line, the needle anode being disposed along the fold-line.

For some applications, the patch is folded along the fold-line, and the fold of the patch along the fold-line facilitates insertion of the needle anode into the skin prior to unfolding of the patch.

For some applications, the patch is folded along the fold-line, the first side of the patch facing outwards.

For some applications, the apparatus further includes an ultrasound transducer interface, configured to removably secure the patch to an ultrasound transducer.

For some applications, the nerve is a sciatic nerve of a subject, and the control circuitry is configured to block the sciatic nerve by driving current between the needle anode and the cathode.

For some applications, the nerve is an occipital nerve of a subject, and wherein the control circuitry is configured to block the occipital nerve by driving current between the needle anode and the cathode.

For some applications, the nerve is a median nerve of a subject, and wherein the control circuitry is configured to block the median nerve by driving current between the needle anode and the cathode.

For some applications, the cathode is an adhesive cathode, configured to adhere the patch to the skin.

There is further provided, in accordance with some applications of the present invention, a method for blocking a nerve under skin of a subject, the method including:

adhering to the skin an adherable patch having a needle anode protruding from a first side of the patch and a cathode disposed on the first side of the patch;

puncturing the skin with the needle anode; and

activating control circuitry on the patch to block the nerve by driving current between the needle anode and the cathode.

For some applications, activating the control circuitry includes activating the control circuitry to block the nerve by driving pulsatile current between the needle anode and the cathode.

For some applications, the method further includes navigating the patch to a site by visualizing the needle anode and the skin through a window in the patch.

For some applications, puncturing the skin with the needle anode includes:

puncturing the skin with the needle anode while the patch is folded; and

subsequently, while the needle anode is inside the skin, unfolding the patch and adhering the patch to the skin.

For some applications, the method further includes removing a tab, the tab being removably coupled to a battery that is disposed in the patch and is configured to be usable upon removal of the tab.

For some applications, the method further includes using ultrasound energy to locate the nerve before the adhering of the patch to the skin.

For some applications, using ultrasound energy to locate the nerve includes navigating the patch to a site of the nerve while the patch is removably secured to an ultrasound transducer.

For some applications, the patch includes a drug for delivery to the subject through the skin of the subject, the method further including iontophoretically administering the drug.

For some applications, iontophoretically administering the drug includes activating a user control coupled to the control circuitry to administer a dose of the drug.

The present invention will be more fully understood from the following detailed description of applications thereof, taken together with the drawings, in which:

### BRIEF DESCRIPTION OF THE DRAWINGS

FIGS. 1-2 are schematic illustrations of an adhesive patch for stimulating a nerve under skin of a subject, in accordance with some applications of the present invention;

FIGS. 3-7 are schematic illustrations of various configurations of the patch of FIGS. 1-2, in accordance with some applications of the present invention;

FIG. 8 is a schematic illustration of control circuitry on the patch, in accordance with some applications of the present invention; and

FIG. 9 is a schematic illustration of the patch adhered to the skin, in accordance with some applications of the present invention.

### DETAILED DESCRIPTION

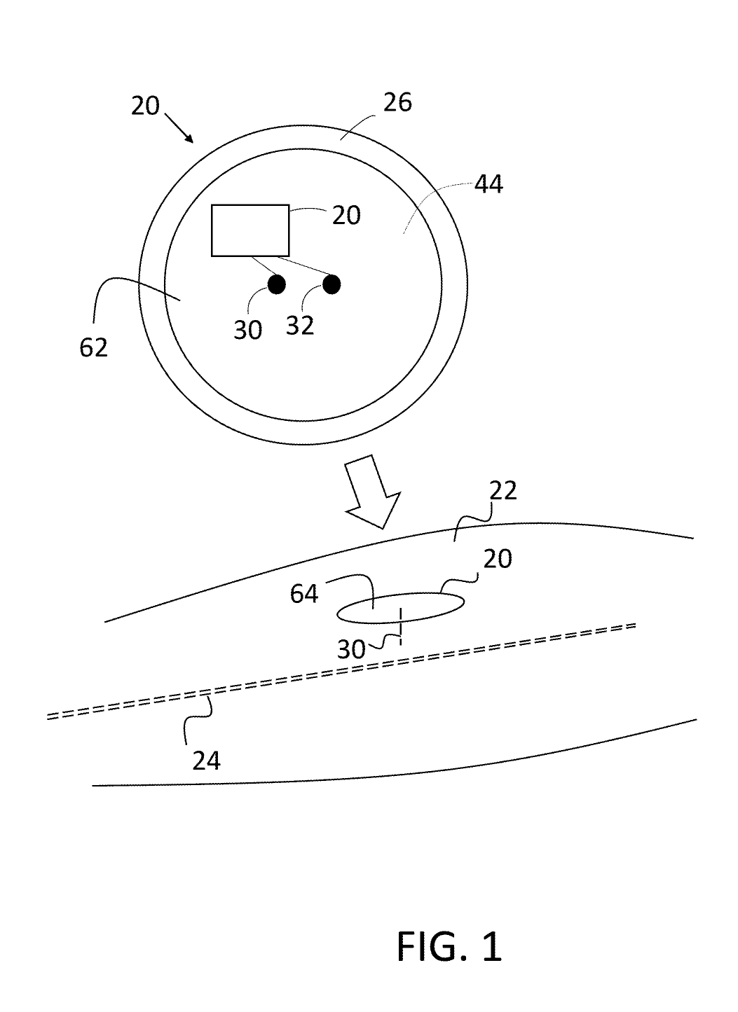

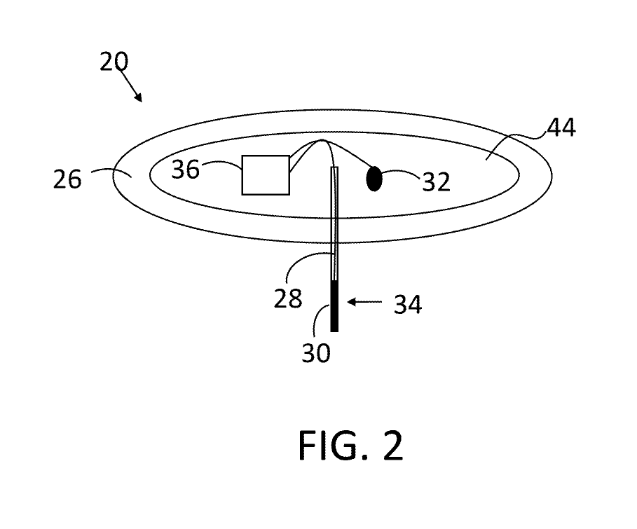

Reference is made to FIGS. 1-2, which are schematic illustrations of an adhesive patch 20, for use with skin 22 of a subject and with a nerve 24 under skin 22. Patch 20 has an adhesive 26 on a first side 62, used to adhere patch 20 to skin 22. Patch 20 comprises a needle 28, protruding from first side 62 of patch 20, an anode 32 disposed on first side 62, and control circuitry 36. Needle 28 is used to puncture skin 22 and comprises a needle cathode 30 disposed on a distal portion 34 of needle 28. Control circuitry 36 is configured to drive pulsatile current between needle cathode 30 and anode 32, thereby stimulating nerve 24. (For some applications, as described hereinbelow, the needle is implemented as a needle anode, and the other electrode is implemented as a cathode.)

Control circuitry 36 is typically configured to drive the pulsatile current at a frequency of at least 20 Hz and/or less than 10 kHz, for example, 20-1000 Hz.

In some applications of the present invention, adhesive 26 is disposed at an edge of patch 20, and patch 20 has a non-adhesive zone 44, in a central region of patch 20. Needle 28 typically protrudes from non-adhesive zone 44 and anode 32 is also typically disposed in non-adhesive zone 44.

In some applications of the present invention, anode 32 is an adhesive anode and/or is disposed in patch 20 where adhesive 26 is disposed (not shown).

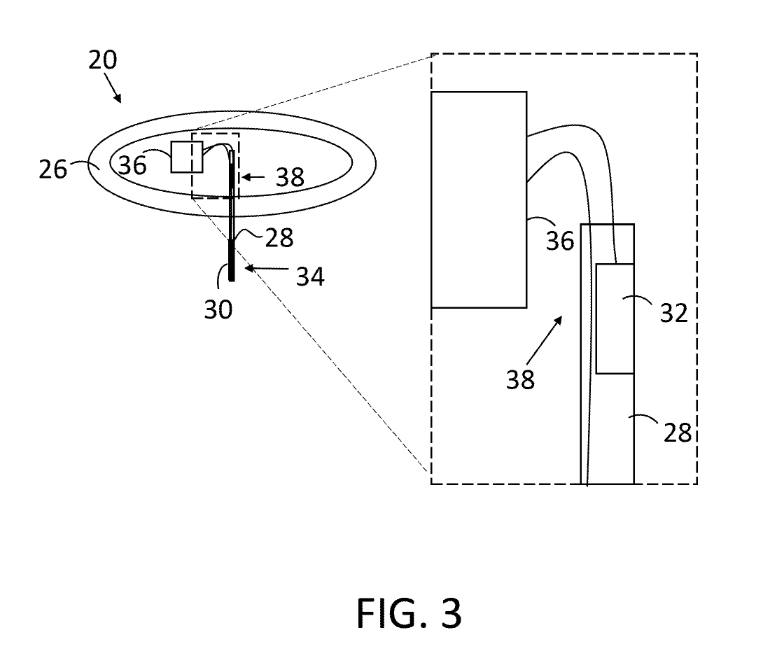

Reference is now made to FIG. 3, which is a schematic illustration of patch 20, in accordance with some applications of the present invention. In some applications, anode 32 is disposed on a proximal portion 38 of needle 28.

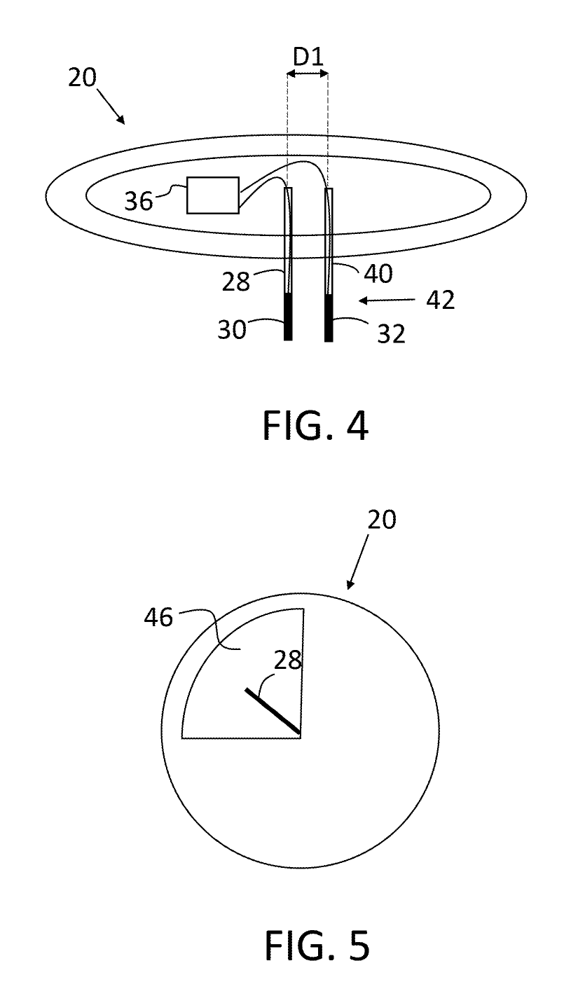

Reference is now made to FIG. 4, which is a schematic illustration of patch 20, in accordance with some applications of the present invention. In some applications of the present invention, needle 28 is a first needle 28, and patch 20 further comprises a second needle 40, also configured to puncture skin 22. Second needle 40 comprises anode 32, which may be disposed on a distal portion 42 of second needle 40. A distance D1 between first needle 28 and second needle 40 is typically at least 5 mm and/or less than 12 mm. First needle 28 and second needle 40 are typically disposed in non-adhesive zone 44.

Reference is now made to FIG. 5, which is a schematic illustration of patch 20, in accordance with some applications of the present invention. In some applications of the present invention, patch 20 is shaped to define at least one window 46. Window 46 is positioned to allow the physician to navigate patch 20 to a site by visualizing needle 28 and skin 22 through window 46 in patch 20.

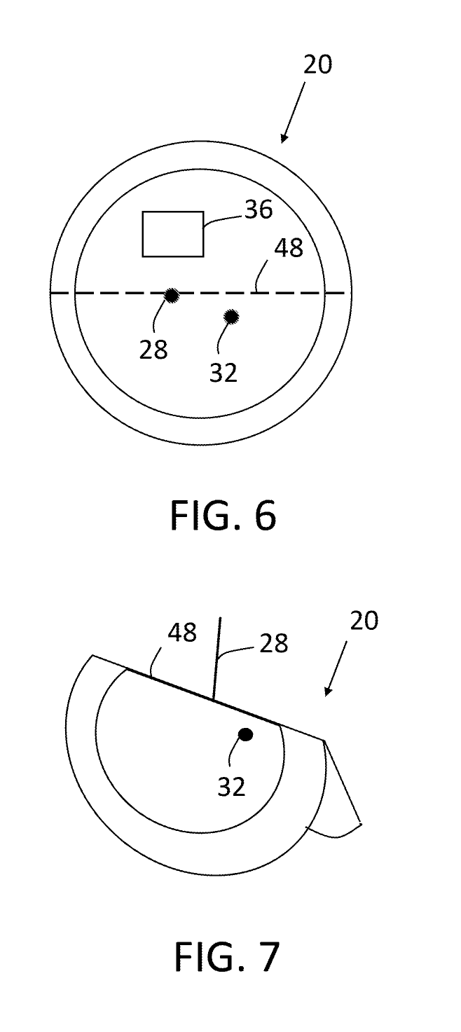

Reference is now made to FIG. 6, which is a schematic illustration of patch 20, in accordance with some applications of the present invention. In some applications of the present invention, patch 20 is shaped to define a fold-line 48, and needle 28 is disposed along fold-line 48. FIG. 7 shows patch 20 folded along fold-line 48, with first side 62 of patch 20 facing outwards, to facilitate insertion of needle 28 into skin 22. The physician punctures skin 22 with needle 28 while patch 20 is folded along fold-line 48, with first side 62 of the patch facing outwards. Subsequently, while needle 28 is inside skin 22, the physician unfolds patch 20 and adheres patch 20 to skin 22.

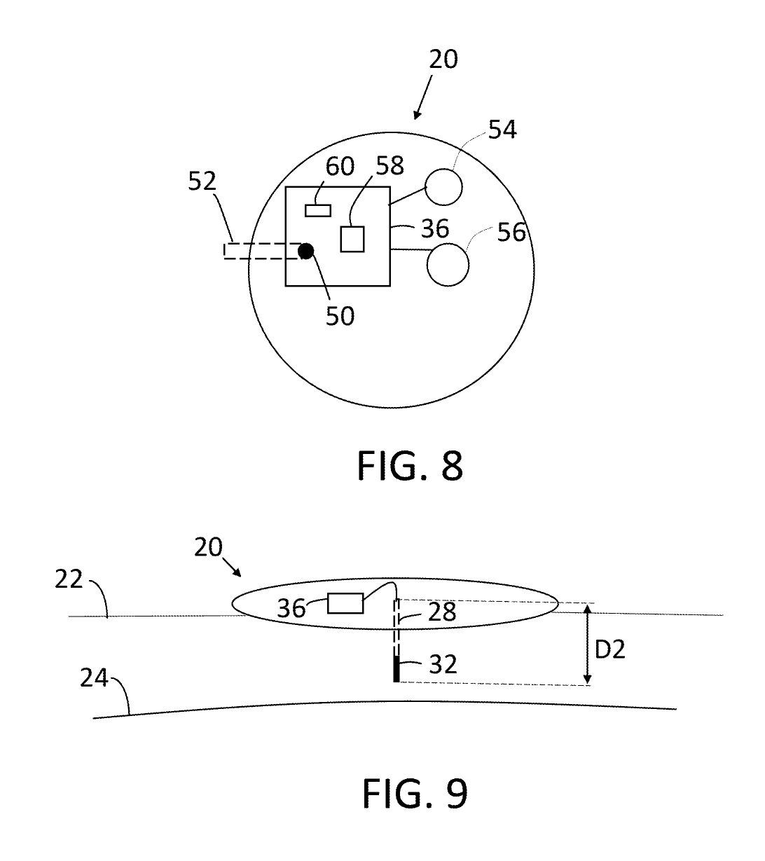

Reference is now made to FIG. 8, which is a schematic illustration of control circuitry 36 on patch 20, in accordance with some applications of the present invention. In some applications, control circuitry 36 comprises a battery 50, which is able to power control circuitry 36 to stimulate nerve 24 for up to a week. Patch 20 further comprises a tab 52 that is removably coupled to battery 50. Battery 50 is configured to be usable upon removal of tab 52.

In some applications of the present invention, control circuitry 36 comprises an indicator 54 disposed on a second side 64 of patch 20, opposite first side 62 of patch 20. Indicator 54 typically indicates when patch 20 is adhered to skin 22 of the subject. For example, indicator 54 may be a visible indicator such as a light emitting diode (LED), or a laser, an audible indicator, or a vibrator.

For some applications, control circuitry 36 comprises at least one physiological sensor 56, e.g., a heart rate sensor or an accelerometer. Control circuitry 36 may be configured to regulate the application of current to nerve 24 in response to data received from physiological sensor 56.

In an application of the present invention, control circuitry 36 comprises a wireless receiver 58, configured to receive data from a remote wireless transmitter (e.g., a cell phone or dedicated external programmer). Control circuitry 36 is configured to alter the pulsatile current in response to data received from the wireless transmitter. In some applications of the present invention, wireless receiver 58 is a Bluetooth™ receiver.

In some applications of the present invention, patch 20 may also be configured to deliver a drug, e.g., a pharmaceutical analgesic, to the subject.

For example, the drug may be iontophoretically administered using cathode 30 when a user control 60, coupled to control circuitry 36, is activated.

In another example, patch 20 consistently administers the drug through skin 22, and control circuitry 36 is used to stimulate nerve 24 when user control 60 is activated.

It is noted that a patch with the ability to provide both drug administration and nerve stimulation can allow the dosage of the administered drug to be reduced. Patch 20 can be worn as an analgesic drug patch, and if the subject feels, at any given point, that the pain medication is insufficient, the subject may activate user control 60 to stimulate nerve 24 for further pain relief.

In some applications of the present invention, patch 20 may not comprise a drug, and is used solely for electrical stimulation of nerve 24.

In some applications of the present invention, patch 20 comprises an ultrasound transducer interface (not shown), configured to removably secure patch 20 to an ultrasound transducer. In some applications of the present invention, patch 20 is removably secured to an ultrasound transducer (not shown) and the physician uses ultrasound energy to locate nerve 24 and navigate patch 20 to a site before adhering patch 20 to skin 22.

Reference is now made to FIG. 9, which is a schematic illustration of patch 20 adhered to skin 22, in accordance with some applications of the present invention. Typically, nerve 24 is any nerve that is 5-6 mm under skin 22.

In some applications of the present invention, nerve 24 is a tibial nerve of the subject, and control circuitry 36 is used to stimulate the tibial nerve by driving pulsatile current between cathode 30 and anode 32. For use with the tibial nerve, length D2 of needle 28 is typically at least 2 mm and/or less than 8 mm long.

In some applications of the present invention, nerve 24 is a sciatic nerve of a subject, and control circuitry 36 is used to stimulate the sciatic nerve by driving pulsatile current between cathode 30 and anode 32.

In some applications of the present invention, nerve 24 is an occipital nerve of a subject, and control circuitry 36 is used to stimulate the occipital nerve by driving pulsatile current between cathode 30 and anode 32. Stimulating the occipital nerve may be used as a treatment for migraine or another condition.

In some applications of the present invention, nerve 24 is a median nerve of a subject, and control circuitry 36 is used to stimulate the median nerve by driving pulsatile current between cathode 30 and anode 32. Stimulating the median nerve may be used as a treatment for carpal tunnel pain or another condition.

It is noted that applications of the present invention as described hereinabove can be implemented in an opposite configuration, wherein the needle, described hereinabove as needle cathode 30, can be implemented as a needle anode, and the second electrode, described hereinabove as anode 32, can be implemented as a cathode. Control circuitry 36 can thus be configured to apply anodal blocking, as is known in the art, by driving current between the needle anode and the cathode, wherein the current, described hereinabove as a pulsatile current having a frequency of 20 Hz-10 kHz, would typically be a non-pulsatile current, or driven at a significantly lower frequency.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with skin of a subject and with a nerve under the skin, the apparatus comprising:
a patch, having an adhesive on a first side of the patch, configured to adhere the patch to the skin;
a needle comprising a needle cathode, protruding from the first side of the patch and configured to puncture the skin;
an anode; and
control circuitry, configured to stimulate the nerve by driving pulsatile current between the needle cathode and the anode.

2. The apparatus according to claim 1, wherein the anode is disposed at the first side of the patch.

3. The apparatus according to claim 1, wherein the anode is disposed on a proximal portion of the needle and the needle cathode is disposed on a distal portion of the needle.

4. The apparatus according to claim 1, wherein the control circuitry comprises a light emitting diode (LED), configured to emit light upon the patch being adhered to the skin of the subject, that is disposed on a second side of the patch opposite to the first side of the patch.

5. The apparatus according to claim 1, wherein the patch is shaped to define at least one window, the window positioned to allow visualizing of the needle cathode during puncturing of the skin by the needle cathode.

6. The apparatus according to claim 1, wherein the patch does not comprise a drug.

7. (canceled)

8. The apparatus according to claim 1, wherein the needle is a first needle, and wherein the apparatus further comprises a second needle, the second needle (a) comprising the anode, (b) protruding from the first side of the patch, and (c) configured to puncture the skin.

9. The apparatus according to claim 8, wherein a distance between the first and second needles is 5-12 mm.

10. (canceled)

11. The apparatus according to claim 1, wherein the nerve is a tibial nerve of the subject, wherein the control circuitry is configured to stimulate the tibial nerve by driving pulsatile current between the needle cathode and the anode, and wherein a length of the needle is 2-8 mm.

12. The apparatus according to claim 1, for use with a wireless transmitter, wherein the control circuitry further comprises a wireless receiver configured to receive data from the wireless transmitter.

13-14. (canceled)

15. The apparatus according to claim 1, wherein the control circuitry comprises at least one physiological sensor.

16-17. (canceled)

18. The apparatus according to claim 1, wherein the patch comprises a drug for delivery to the subject through the skin of the subject.

19. The apparatus according to claim 18, wherein the drug is an analgesic.

20. (canceled)

21. The apparatus according to claim 18, wherein (a) the patch constantly administers the drug through the skin of the subject when the patch is adhered to the skin, and (b) the control circuitry comprises a user control and is configured to stimulate the nerve upon the user control being activated.

22. The apparatus according to claim 1, wherein the adhesive is disposed at an edge of the patch, and wherein the patch has a non-adhesive zone on the first side of the patch, in a central region of the patch.

23-24. (canceled)

25. The apparatus according to claim 1, wherein the control circuitry comprises a battery configured to power the control circuitry to stimulate the nerve for 1-7 days.

26. (canceled)

27. The apparatus according to claim 1, wherein the patch is shaped to define a fold-line, the needle cathode being disposed along the fold-line.

28. The apparatus according to claim 27, wherein the patch is folded along the fold-line, and wherein the fold of the patch along the fold-line facilitates insertion of the needle cathode into the skin prior to unfolding of the patch.

29. The apparatus according to claim 27, wherein the patch is folded along the fold-line, the first side of the patch facing outwards.

30. The apparatus according to claim 1, further comprising an ultrasound transducer interface, configured to removably secure the patch to an ultrasound transducer.

31-86. (canceled)
